(19) United States
(12) Patent Application Publication
(10) Pub. No.: US 2026/0315548 A1
(43) Pub. Date: Oct. 15, 2026

(54) AN ANTIBIOTIC AND A MICROORGANISM HAVING BILE ACID 7alpha-DEHYDROXYLATION ACTIVITY FOR TREATING NON-INFECTIOUS DISEASES

(71) Applicant: ECOLE POLYTECHNIQUE FEDERALE DE LAUSANNE (EPFL), Lausanne (CH)

(72) Inventors: Kristina SCHOONJANS, Lausanne (CH); Alessia PERINO, Lausanne (CH); Antoine JALIL, Lausanne (CH)

(21) Appl. No.: 19/475,216

(22) PCT Filed: Apr. 18, 2024
(86) PCT No.: PCT/EP2024/060482

§ 371 Date: Oct. 14, 2025

(30) Foreign Application Priority Data
Apr. 20, 2023 (EP) 23168848.2

(51) Int. Cl. A61K 35/742; A61K 9/00; A61K 31/4164; A61K 31/7048; A61K 38/14; A61P 1/00; A61P 3/00

(52) U.S. Cl. CPC A61K 35/742; A61K 9/0053; A61K 31/4164; A61K 31/7048; A61K 38/14; A61P 1/00; A61P 3/00

(57) ABSTRACT

The present invention relates to one or more antibiotic(s) that disrupt(s) the microbial balance of the gut and a microorganism having bile acid 7α-dehydroxylation activity for use in the treatment of a non-infectious disease in a subject.

The present invention relates to one or more antibiotic(s) that disrupt(s) the microbial balance of the gut and a microorganism having bile acid 7α-dehydroxylation activity for use in the treatment of a non-infectious disease in a subject.

In this specification, a number of documents including patent applications, research articles and manufacturer's manuals are cited. The disclosure of these documents, while not considered relevant for the patentability of this invention, is herewith incorporated by reference in its entirety. More specifically, all referenced documents are incorporated by reference to the same extent as if each individual document was specifically and individually indicated to be incorporated by reference.

The gut microbial community produces a plethora of metabolites controlling host physiology. Several of these function as host signaling molecules, including secondary bile acids (BAS)¹. BAs are initially generated in the liver as primary BAs (cholic acid (CA) and chenodeoxycholic acid (CDCA) in humans and CA, CDCA, and muricholic acid (MCA) in mice), and secreted into the intestine upon food intake, where they act as lipid-solubilizing agents^{2,3}. In the intestine, primary BAs are transformed by the gut microbiota into a variety of secondary BAs with different bioactivities⁴⁻⁶. The most abundant transformation in the colon involves the removal of the hydroxyl group at the C7 position of the primary BAS CA and CDCA (i.e. 7α-dehydroxylation, 7α-DH-ion) to generate the secondary BAs deoxycholic acid (DCA) and lithocholic acid (LCA), respectively^{7,8}. This reaction is carried out by bacteria harboring a bile acid-inducible (bai) operon composed of eight different genes required for the multi-step BA 7α-DH-ion reaction^{9,10}, with Clostridium scindens(C. scindens) being the best studied and characterized⁷. BAS, including the 7α-dehydroxylated (7α-DH-ed) forms, can escape from the enterohepatic organs into the systemic circulation and function as bona fide hormones that control whole-body metabolism^{2,11}. In particular, 7α-DH-ed BAs are the most potent endogenous agonists of the G protein-coupled BA receptor 1 (GPBAR1), also known as Takeda G protein-coupled receptor (TGR5)¹², and activation of this signaling pathway by endogenous or TGR5 selective semi-synthetic BAs13 controls glucose and energy homeostasis in preclinical models of diabesity¹⁴⁻²², as well as in healthy subjects²³. Several mechanisms underlie these phenotypes, ranging from beiging^{17,24,25} and suppression of chronic inflammation in resident macrophages of white adipose tissue (WAT)²⁶, to stimulation of fat oxidation in brown adipose tissue (BAT) and muscle^{15,23,27}. Activation of TGR5 also improves muscle function by enhancing hypertrophy²⁸ and by increasing glucose utilization²⁹, mechanisms that may further contribute to the TGR5-dependent metabolic benefits. Importantly, TGR5 signaling improves glycemic control by increasing the secretion of the insulinotropic hormone glucagon-like peptide-1 (GLP-1) from intestinal enteroendocrine L-cells^{14,30-33} and more recent studies have shown that this axis also increases the number of L-cells in the distal parts of the intestine by enhancing the proliferation and specification of intestinal stem cells (ISCs) 34,35. These findings suggest that 7α-DH-ed BAs are powerful boosters of GLP-1 signaling, which coordinates postprandial insulin secretion and satiety^{36,37} and is a first-line target in the treatment for type-2 diabetes (T2D)³⁷. The recent discovery that BAs reach the mouse brain shortly after a meal to suppress food intake through TGR5 activation in the hypothalamus^{22,38} further supports the notion that BAs and their receptors act as essential integrators of nutrient availability to govern various regulatory circuits involved in whole-body energy and metabolic homeostasis³⁹. Although the beneficial metabolic effects of TGR5 activation are well characterized, TGR5 agonists are not yet in the clinic and strategies aimed at activating this signaling pathway by modulating the gut microbiota could represent a novel therapeutic approach.

T2D and its comorbidities are growing metabolic disorders with devastating socio-economic consequences40. Obesity is considered the major risk factor for the development of T2D⁴¹ and strategies focused on weight management are at the forefront of the prevention of this disease⁴². In this context, bariatric surgery is one of the most effective treatments to combat diabesity, in which the modulation of signals emanating from the gut seems to play a pivotal role^{43,44}. Gut hormone levels, with the insulinotropic GLP-1 in first line, are markedly increased in rodent models and patients undergoing bariatric surgery, and confer, among other effects, satiety in the brain^{36,45}. Bariatric surgery is also effective in restoring the gut commensal microbial community, which has been shown to be dysbiotic in obesity⁴⁶⁻⁴⁸ and T2D^{48,49}. Fecal microbiota transplantation, especially when combined with lifestyle intervention, has been proven to be beneficial in obese patients with T2D⁵⁰. Thus, better knowledge of the alterations in the composition of the gut microbiota and its derived metabolites, including BAS, may open up new therapeutic options for the treatment of metabolic disorders, including obesity, T2D, nonalcoholic fatty liver disease (NAFLD) and metabolic dysfunction-associated fatty liver disease (MAFLD).

Gut dysbiosis is also frequently found in patients with inflammatory bowel diseases (IBD)^{51,52}. Metabolic studies have identified that IBD patients are characterized by major changes in the stool BA composition, with significant enrichment of primary BAs and a decrease in secondary BAs that may be a contributing factor in the progression of the disease^{53,54}. IBD, which includes Crohn's disease (CD) and ulcerative colitis (UC), is a chronic relapsing disorder characterized by inflammation of the digestive tract. Although the etiology of IBD is still unclear, several factors are known to contribute to its incidence, including genetic predisposition, environmental factors, and gut dysbiosis^{51,55}. IBD is characterized by a major shift in the gut microbiome and a decrease in its diversity which result in alterations in bacterial metabolites, including BAs⁵³. A major complication of IBD is ulceration of the intestinal epithelium, especially during disease flares⁵⁶ and preserving the homeostasis of the intestinal epithelium appears crucial to prevent disease progression and tissue damage. The intestinal epithelium is a highly plastic and dynamic tissue that renews itself every 2-5 days⁵⁷ through the action of tissue-resident ISCs, which are responsible for replenishing the cells lining the gut epithelium⁵⁸. In cases of recurrent epithelial damage due to inflammatory flares, the regenerative capacity of ISCs is essential for rebuilding the epithelium and restoring intestinal function⁵⁹. While immunosuppressive drug treatment has significantly improved the health of IBD patients in the last decades, mucosal healing has long been neglected and has only recently emerged as another possible key therapeutic strategy^{60,61}. However, the controlled targeting of tissue regeneration has not yet been achieved. Recently, we have demonstrated that 7α-DH-ed BAs could be leveraged to modulate this process through activation of TGR5 expressed in ISCs³⁴.

Altogether, these observations led us to hypothesize that a restoration of the 7α-DH-ed BA levels through an elevation in the 7α-DH-ion capacity of the gut microbiota in vivo may be beneficial on multiple fronts. On the one hand, increasing the levels of 7α-DH-ed BAs could contribute to improved whole-body energy and metabolic homeostasis; on the other hand, restoring the physiological levels of these gut-derived metabolites could ameliorate mucosal healing in IBD. Therefore, strategies aimed at restoring 7α-DH-ed BA levels may represent novel prophylactic therapies to counteract metabolic disorders and IBD progression.

In view of the above, the present invention aims at the provision of novel treatment options for diseases that are at least in part caused by altered gut microbiota and/or can be treated by altering the gut microbiota.

Accordingly, the present invention relates to one or more antibiotic(s) that disrupt(s) the microbial balance of the gut and a microorganism having bile acid 7α-dehydroxylation activity for use in the treatment of a non-infectious disease in a subject.

Also described herein is a corresponding method of treatment comprising the administration of one or more antibiotic(s) that disrupt(s) the microbial balance of the gut and a microorganism having bile acid 7α-dehydroxylation activity to a subject having a non-infectious disease.

An antibiotic is a type of antimicrobial substance being active against bacteria. It is the most important type of antibacterial agent for fighting bacterial infections, and antibiotic medications are widely used in the treatment and prevention of such infections. They may either kill or inhibit the growth of bacteria.

The one or more antibiotic(s) to be used herein is/are capable of disrupting the microbial balance of the gut upon their administration to the subject to be treated.

The gut microbiota is a group of microorganisms, mainly bacteria that are deposited throughout the entire gut. The trillions of bacteria, fungi and other microbes that live in our gut are essential contributors to good health. They break down toxins, manufacture some vitamins, essential amino acids and metabolites, and form a barrier against invaders. Each subject has an entirely unique gut microbiome that is originally determined by the genetics of a subject and then also by environmental exposures, such as birth, nutrition, and other environmental factors. This entirely unique gut microbiome is designated “microbial balance” herein. The gut microbiome of each subject consists of microbes that are both helpful and potentially harmful. Most are symbiotic (where both the subject and microbiota benefit) and some are pathogenic (promoting disease of a subject). In a healthy body, pathogenic and symbiotic microorganisms coexist without problems. But if there is a disturbance in that balance, for example, brought on by a disease, dysbiosis occurs. Dysbiosis (also called dysbacteriosis) is characterized by a disruption of the microbiome resulting in an imbalance in the microbiome homeostasis.

Hence, upon the administration of the one or more antibiotic(s) to the subject to be treated, the gut microbiome will be remodeled and selective classes of bacteria will be killed or their growth will be inhibited, by the effect of the one or more antibiotic(s).

The microorganism, or microbe, is an organism of microscopic size, which may exist in its single-celled form or as a colony of cells. The major groups of microorganisms are bacteria, archaea, fungi (yeasts and molds), algae, protozoa and viruses.

The microorganism to be employed in accordance with the invention has bile acid 7α-dehydroxylation activity and is therefore also referred to herein as 7α-dehydroxylating microorganism.

Bile acids are steroid acids found predominantly in the bile of mammals and other vertebrates. Diverse bile acids are synthesized in the liver and are conjugated with taurine or glycine residues. Primary and secondary bile acids are distinguished in the art. Primary bile acids (cholic acid and chenodeoxycholic acid in humans) are synthesized in the liver, in particular in the hepatocytes, via cytochrome P450-mediated oxidation of cholesterol via a multi-step enzymatic process. In humans, approximately 600 mg of bile acids are synthesized daily to replace those lost in the feces. Once secreted into the lumen of the intestine, primary bile acids can be modified by gut bacteria to form secondary bile acids. During this process, glycine and taurine groups are removed by microorganisms carrying bile salt hydrolase activity and transform the conjugated bile acids back into unconjugated primary bile acids, which subsequently serve as substrates for the 7α-dehydroxylating microorganisms, yielding 7α-dehydroxylated secondary bile acids. Hence, cholic acid and chenodeoxycholic acid can be converted into deoxycholic acid and lithocholic acid, respectively.

The secondary bile acids formed by gut microbiota from primary bile acids have wide-ranging effects on the metabolism of a subject and play an important role in health and disease. The enzymatic pathway carrying out 7α-dehydroxylation is restricted to a narrow phylogenetic group of commensal bacteria, the best-characterized of which is Clostridium scindens. While number of bacterial species having 7α-dehydroxylation activity are known and will be described herein below, also strategies for identifying further such bacterial species are available, for example, from Vital et al (2019) Comput Struct Biotechnol J. 2019; 17: 1016-1019.

Non-infectious diseases are diseases that are not caused by germs or microorganisms, such as bacteria, viruses, fungi and parasites. The non-infectious disease is preferably a noncommunicable disease (NCD), also known as chronic disease. Such a disease is the result of one or more of genetic, physiological, environmental and behavioral factors. It is generally characterized by a long duration. The non-infectious disease is preferably characterized by altered, e.g. impaired gut microbial activity.

The one or more antibiotic(s) that disrupt(s) the microbial balance of the gut is/are preferably to be administered orally and the microorganism having bile acid 7α-dehydroxylation activity is preferably to be administered orally or rectally.

The one or antibiotic(s) that disrupt(s) the microbial balance of the gut and the microorganism having bile acid 7α-dehydroxylation is/are preferably formulated as a pharmaceutical kit. A pharmaceutical kit generally refers to a packaged set of related pharmaceuticals/drugs and/or pharmaceutical/drug delivery devices to be used for a particular medical treatment. The pharmaceutical kit preferably further includes the required documentation for the kit components and/or instructions how to use the kit.

As is evident from the appended examples, 7α-dehydroxylating bacteria in combination with antibiotic treatment is particularly useful for treating non-infectious diseases associated with an altered gut microbiota and/or that can be treated by altering the gut microbiota. The advantage of the therapeutic progress of the invention is the combination of the 7α-dehydroxylating bacteria with one or more antibiotic(s) that disrupt(s) the microbial balance of the gut, in order to allow an optimal engraftment of the exogenously administered microorganisms. Antibiotics are usually used to treat bacterial infections and not non-infectious diseases. It is known that antibiotics have a detrimental impact on the gut microbiota because they not only kill and/or inhibit the infectious bacteria but likewise the commensal bacteria that reside in the gut and play a fundamental role in human health. Indeed, the gut microbiota prevents pathogen colonization, provides essential nutrients and bioactive metabolites, and contributes to the regulation of multiple host processes, including gut immunity and energy homeostasis. These commensal bacteria have different sensitivities of antibiotics. Some bacteria in the community might disappear and others might overgrow, so that the balance becomes different as a consequence of the treatment by the antibiotics. The appended examples show that one or more antibiotic(s) that disrupt(s) the microbial balance of the gut favor the colonization and growth of the 7α-dehydroxylating bacteria in the gut while 7α-dehydroxylating bacteria cannot colonize and grow as well as without this/these antibiotic(s). Hence, it was found herein that the treatment of non-infectious diseases with 7α-dehydroxylating bacteria can be improved by the combination with one or more antibiotic(s) that disrupt(s) the microbial balance of the gut.

In accordance with a preferred embodiment the one or more antibiotic(s) is/are to be administered in a pre-treatment to disrupt the microbial balance of the gut before the microorganism having bile acid 7α-dehydroxylation activity is to be administered to the subject.

The use of the one or more antibiotic(s) to disrupt the microbial balance of the gut is particularly advantageous as pre-treatment since it enhances the gut susceptiveness for the microorganism having bile acid 7α-dehydroxylation activity already before these bacteria reach the gut.

In accordance with a more preferred embodiment the one or more antibiotic(s) is/are to be administered to disrupt the microbial balance of the gut for at least 1 day, preferably at least 3 days and most preferably at least 7 days before the microorganism having bile acid 7α-dehydroxylation activity is to be administered, preferably orally.

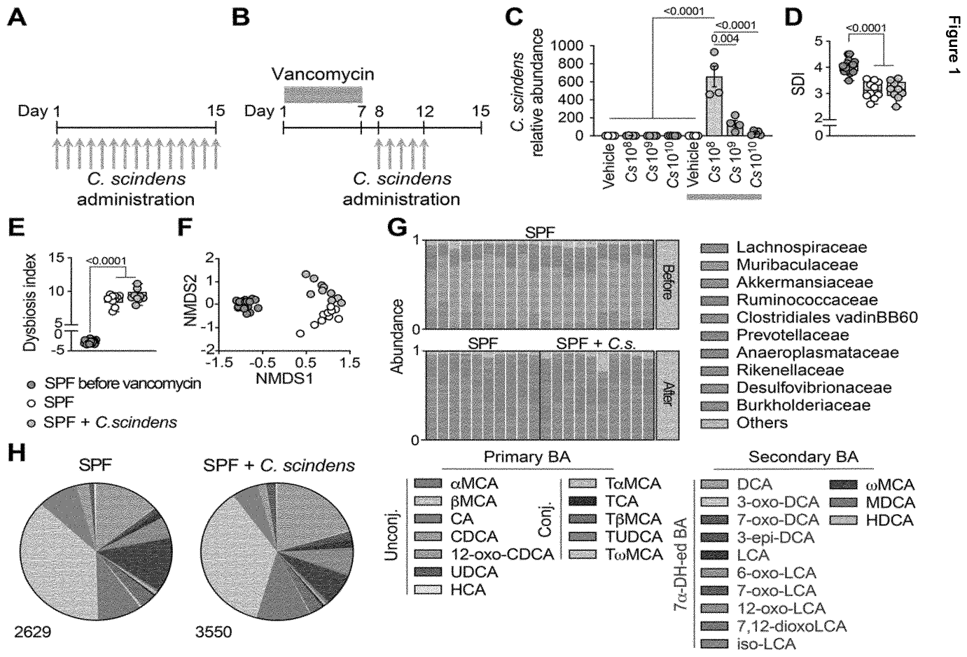

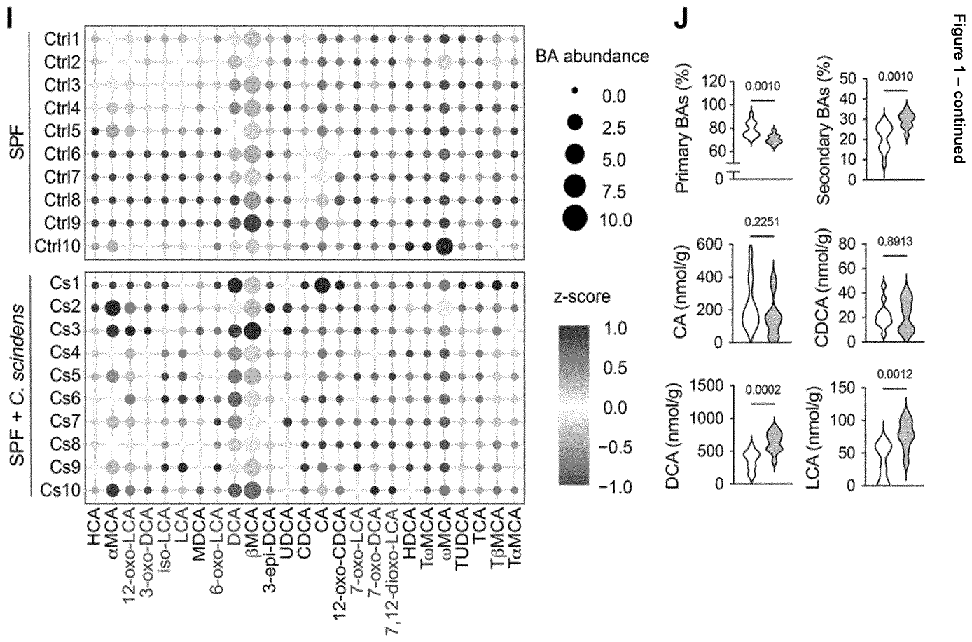

The above more preferred embodiment is directed to the duration of the antibiotic pre-treatment prior to the administration of the microorganism having bile acid 7α-dehydroxylation activity. With increasing preference of the duration time, the gut becomes more and more susceptible for the 7α-dehydroxylating bacteria engraftment. It can be taken from FIG. 1 to 3 that in the example mice were pre-treated with the antibiotic vancomycin for 7 days.

In accordance with a further preferred embodiment the one or more antibiotic(s) is/are selected from antibiotics against gram-positive bacteria, preferably from antibiotics being effective against Clostridioides difficile, and most preferably from vancomycin, fidaxomicin, and metronidazole.

In accordance with this preferred embodiment the antibiotic(s) is/are selected such that the antibiotic(s) is/are particular effective for killing and/or inhibiting bacteria that show taxonomic proximity to the 7α-dehydroxylating bacteria, so that susceptiveness is ideally obtained in the same ecological gut niche that is needed for the colonization and growth of the 7α-dehydroxylating bacteria in the gut.

7α-dehydroxylating bacteria are generally gram-positive and in particular Clostridioides. In the appended examples Clostridium scindens is used as the 7α-dehydroxylating bacterium. In view of the taxonomic proximity of Clostridium scindens and the disease-causing Clostridioides difficile (Eubacteriales order) antibiotic(s) being effective against Clostridioides difficile is/are particularly preferred. Vancomycin, fidaxomicin, and metronidazole are known to be effective against Clostridioides difficile. Clostridioides difficile causes serious diarrheal infections and may also cause colon cancer.

In accordance with a further preferred embodiment the one or more antibiotic(s) is/are to be administered (a) in an amount of 6 to 10 mg/kg, preferably 7 to 8.5 mg/kg of body weight of the subject to be treated, or (b) in an amount of 100 to 150 mg, most preferably about 125 mg 4 times daily, preferably orally.

The above dosages of antibiotic(s) (such as vancomycin, as used in the appended examples) are typically administered to human subjects to treat a bacterial infection with gram-positive bacteria, such as Clostridioides and in particular Clostridioides difficile. It can be expected that the above dosages are likewise preferred dosages for disrupting the microbial balance of the gut.

In the case the one or more antibiotic(s) is one antibiotic (such as vancomycin, as used in the appended examples) the antibiotic is to be administered (a) in an amount of 6 to 10 mg/kg, preferably 7 to 8.5 mg/kg of body weight of the subject to be treated, or (b) in an amount of 100 to 150 mg, most preferably about 125 mg 4 times daily, preferably orally. In the case the one or more antibiotic(s) are two or more antibiotics the antibiotics are preferably to be administered (a) in a total amount of 6 to 10 mg/kg, preferably 7 to 8.5 mg/kg of body weight of the subject to be treated, or (b) in a total amount of 100 to 150 mg, most preferably about 125 mg 4 times daily, preferably orally.

In accordance with another preferred embodiment the microorganism is a bacterium, preferably a bacterium of the class Clostridia and most preferably Clostridium scindens, Clostridium hylemonae, Peptacetobacter (Clostridium) hiranonis, or Proteocatella sphenisci.

A number of microorganisms, in particular bacteria having bile acid 7α-dehydroxylation activity are known in the art. Many on these bacteria can be found in class Clostridia. Non-limiting but preferred examples are Clostridium scindens, Clostridium hylemonae, Peptacetobacter (Clostridium) hiranonis, and Proteocatella sphenisci.

Within this list Clostridium scindens is most preferred since Clostridium scindens is used in the appended examples. Clostridium scindens is a species that is found in the human gut. It is known that Clostridium scindens becomes established in the human colon, and its presence is associated with resistance to infection by Clostridioides difficile, due to the production of secondary bile acids via 7α-dehydroxylation activity which inhibit the growth of Clostridioides difficile. The treatment of an Clostridioides difficile infection by antibiotics and/or Clostridium scindens is described in U.S. Pat. No. 11,471,495 and Buffie et al. (2015), Nature, 517: 205-208.

Also Clostridium hylemonae and Peptacetobacter (Clostridium) hiranonis can be found in the human gut and both of them produce secondary bile acids which inhibit the growth of Clostridioides difficile via 7α-dehydroxylation activity. Proteocatella sphenisci can be found in the gut of penguin guano and is also known to produce secondary bile acids via 7α-dehydroxylation activity (Lee et al. (2022), Gut Microbes. 2022; 14(1): 2132903).

In accordance with a more preferred embodiment the bacterium is in a proliferative state, an attenuated state or is a bacterial spore, preferably in a proliferative state or is a bacterial spore.

A bacterium being in proliferative state is capable of growing by cell duplication. As single-cell organisms bacteria rely on cell duplication to proliferate. Bacterial cell proliferation is generally highly efficient, both because bacteria grow fast and multiply with a low failure rate.

Attenuated bacteria (or live attenuated bacteria) are “weakened” versions of infectious bacteria. In the present case they are modified, so that they cannot cause harm or disease in the body but are still able to produce secondary bile acids via 7α-dehydroxylation activity. Bacteria are attenuated (weakened) in a laboratory, usually by repeated culturing.

Bacterial species have different copying mechanisms with selective harsh environmental conditions. One of the most common coping mechanisms for bacteria is spore formation to protect themselves against ecological degrading agents. Bacterial spores are the most dormant form of bacteria since they exhibit minimal metabolism and respiration, as well as reduced enzyme production. Typically, gram-positive bacteria are best known for producing intracellular spores called endospores as a survival mechanism.

In accordance with a different preferred embodiment the non-infectious disease is an inflammatory disease or a metabolic disease.

In accordance with a more preferred embodiment, the inflammatory disease is inflammatory bowel disease (IBD), and is preferably Crohn's disease and/or ulcerative colitis.

In accordance with another more preferred embodiment the metabolic disease is obesity, metabolic dysfunction-associated fatty liver disease (MAFLD), non-alcoholic fatty liver disease (NAFLD) or diabetes, wherein the diabetes is preferably type-2 diabetes.

Example 1 herein below demonstrates in a mouse model that the combination of one or more antibiotic(s) that disrupt(s) the microbial balance of the gut (in particular in the form of a pre-treatment) and a microorganism having bile acid 7α-dehydroxylation activity is useful for treating the inflammatory disease IBD. Example 2 herein below demonstrates in a mouse model that the combination is likewise useful for treating the metabolic disease obesity. Inflammatory diseases include a vast array of disorders and conditions that are characterized by inflammation. Examples include allergy, asthma, autoimmune diseases, coeliac disease, glomerulonephritis, hepatitis, IBD, reperfusion injury and transplant rejection. Preferred examples of inflammatory diseases are Crohn's disease and/or ulcerative colitis since they are forms of IBD. The term IBD describes a group of disorders in which the intestines become inflamed. It has often been thought of as an autoimmune disease, but research suggests that the chronic inflammation may not be due to the immune system attacking the body itself. The successful treatment of IBD is shown in Example 1.

Metabolism is the process in the body being used to produce energy from the food one eats. Food is made up of proteins, carbohydrates, and fats. Chemicals in the digestive system break the food parts down into sugars and fatty acids to produce energy. A metabolic disorder occurs when abnormal chemical reactions in the body disrupt this process. The metabolic disorder to be treated herein is preferably characterized by abnormal chemical reactions in the gut, more preferably by abnormal or insufficient secondary bile acid formation. Non-limiting but preferred examples of the metabolic disorder are obesity, MAFLD; NAFLD or diabetes, wherein the diabetes is preferably type-2 diabetes, wherein obesity is most preferred in view of Example 2.

MAFLD is the term for a range of conditions caused by a build-up of fat in the liver. MAFLD develops in 4 main stages. The main stages of MAFLD and NAFLD are: (1) Fatty liver (steatosis)—a largely harmless build-up of fat in the liver cells that may only be diagnosed during tests carried out for another reason, (2) Metabolic dysfunction-associated steatohepatitis (MASH, or steatohepatitis)/Non-alcoholic steatohepatitis (NASH, or steatohepatitis)—a more serious form of MAFLD or NAFLD, where the liver has become inflamed. (3) Fibrosis—where persistent inflammation causes scar tissue in the liver and nearby blood vessels, but the liver is still able to function normally. (4) Cirrhosis-the most severe stage, occurring after years of inflammation, where the liver shrinks and becomes scarred and lumpy; this damage is permanent and can lead to liver failure and liver cancer (hepatocellular carcinoma).

In accordance with a further preferred embodiment the microorganism having bile acid 7α-dehydroxylation activity is to be administered in an amount of between 10⁵ and 10⁹ colony forming units (CFUs), preferably 10⁷ and 10⁹ CFUs per day and most preferably about 10⁸ CFUs per day, preferably orally or rectally.

It can be taken from FIG. 1C of the application that mice were treated with different amounts of the microorganism having bile acid 7α-dehydroxylation activity Clostridium scindens; 10⁸, 10⁹ and 10¹⁰ colony forming units (CFUs). In this connection it was surprisingly found that not the highest amount of 10¹⁰ CFUs resulted in the greatest treatment success but instead and not as expected beforehand the lowest amount of 10⁸ CFUs. For unknown reasons the lower amount of 10⁸ CFUs results in a better colonization of the gut and better bile acid 7α-dehydroxylation activity than the 100-fold higher amount of 10¹⁰ CFUs.

It can be assumed that 10⁸ CFUs in mouse per day correspond to 10⁵ and 10⁹ colony forming units (CFUs), preferably 10⁷ and 10⁹ CFUs per day and most preferably about 10⁸ CFUs per day in humans. In accordance with a further preferred embodiment the microorganism having bile acid 7α-dehydroxylation activity is to be administered for at least 1 day, preferably at least 2 days, more preferably at least 3 days, even more preferably 4 days and most preferably at least 5 days, preferably orally or rectally.

While it is sufficient and preferred to administer the microorganism having bile acid 7α-dehydroxylation activity once on one day, the administration may also be repeated with increasing preference for two or more days, such as at least 2 days, at least 3 days, at least 4 days or at least 5 days.

In accordance with a related preferred embodiment the microorganism having bile acid 7α-dehydroxylation activity is to be administered together with a microorganism having bile salt hydrolase activity, wherein the microorganism having bile salt hydrolase activity is preferably a bacterium, more preferably a bacterium of the genus Faecalibacterium, Bacteroides, Lactococcus or Mediterraneibacter, and most preferably Faecalibacterium prausnitzii, Bacteroides dorei, Lactococcus lactis, Faecalibaculum rodentium or Ruminococcus gnavus.

7α-dehydroxylating bacteria are not the only bacterial species being important for the production of secondary bile acids in the gut. Indeed, primary bile acids produced by the liver are conjugated with an amino acid (glycine or taurine) before being secreted into the gut, where the amino acid entity needs to be removed before further transformation (e.g. 7α-dehydroxylation). This deconjugation reaction is carried out by bacteria carrying a bile salt hydrolase enzyme⁶². Therefore, co-administration of a bacterial strain carrying a bile salt hydrolase enzyme and bacteria capable of 7α-dehydroxylating bile acids is assumed to further increase the levels of 7α-dehydroxylated bile acids and, thus, the treatment of non-infectious diseases according to the invention.

A number of microorganisms having bile salt hydrolase activity are known in the prior art, such as bacteria, in particular bacteria of the genus Faecalibacterium, Bacteroides, Lactococcus and Mediterraneibacter. Non-limiting but preferred examples are Faecalibacterium prausnitzii, Bacteroides dorei, Lactococcus lactis, Faecalibaculum rodentium and Ruminococcus gnavus. Among this list Faecalibacterium prausnitzii and/or Bacteroides dorei and/or Ruminococcus gnavus is/are preferred. For this reason, a particular preferred means to further improve the therapeutic approach of the invention is to co-administer Clostridium scindens and Faecalibacterium prausnitzii, or Clostridium scindens and Bacteroides dorei, or Clostridium scindens and Ruminococcus gnavus.

For the co-administration, preferably between 10⁴ and 10⁸ CFUs and most preferably between 10⁴ and 10⁵ CFUs for each bacterium are to be co-administered.

In accordance with a further preferred embodiment the bile acid 7α-dehydroxylation activity is capable of dehydroxylating the primary bile acids cholic acid (CA) and/or chenodeoxycholic acid (CDCA) into the secondary bile acids deoxycholic acid (DCA) and/or lithocholic acid (LCA), respectively.

In the gut, bile acids interact with the gut microbiome, which performs a wide variety of metabolic modifications including the conversion of primary to secondary bile acids. These primary bile acids must first be deconjugated through the action of bile salt hydrolase and are then converted into secondary bile acids through a multi-step process known as 7α-dehydroxylation. Deoxycholic acid (DCA) and lithocholic acid (LCA) are the most abundant secondary bile acids and are derived from the primary bile acids cholic acid (CA) and chenodeoxycholic acid (CDCA), respectively, via 7α-dehydroxylation.

In accordance with a still further preferred embodiment the subject to be treated is a mammal, preferably human.

The mammal may be a mouse, rat, hamster, guinea pig, rabbit, dog, cat, sheep, pig, goat, cattle, horse, monkey, ape, or human and is most preferably a human.

As regards the embodiments characterized in this specification, in particular in the claims, it is intended that each embodiment mentioned in a dependent claim is combined with each embodiment of each claim (independent or dependent) said dependent claim depends from. For example, in case of an independent claim 1 reciting 3 alternatives A, B and C, a dependent claim 2 reciting 3 alternatives D, E and F and a claim 3 depending from claims 1 and 2 and reciting 3 alternatives G, H and I, it is to be understood that the specification unambiguously discloses embodiments corresponding to combinations A, D, G; A, D, H; A, D, I; A, E, G; A, E, H; A, E, I; A, F, G; A, F, H; A, F, I; B, D, G; B, D, H; B, D, I; B, E, G; B, E, H; B, E, I; B, F, G; B, F, H; B, F, I; C, D, G; C, D, H; C, D, I; C, E, G; C, E, H; C, E, I; C, F, G; C, F, H; C, F, I, unless specifically mentioned otherwise.

Similarly, and also in those cases where independent and/or dependent claims do not recite alternatives, it is understood that if dependent claims refer back to a plurality of preceding claims, any combination of subject-matter covered thereby is considered to be explicitly disclosed. For example, in case of an independent claim 1, a dependent claim 2 referring back to claim 1, and a dependent claim 3 referring back to both claims 2 and 1, it follows that the combination of the subject-matter of claims 3 and 1 is clearly and unambiguously disclosed as is the combination of the subject-matter of claims 3, 2 and 1. In case a further dependent claim 4 is present which refers to any one of claims 1 to 3, it follows that the combination of the subject-matter of claims 4 and 1, of claims 4, 2 and 1, of claims 4, 3 and 1, as well as of claims 4, 3, 2 and 1 is clearly and unambiguously disclosed.

The figures show.

FIG. 1. C. scindens effectively colonizes vancomycin-treated SPF mice and changes their fecal bile acid pool under basal conditions. (A and B) Workflows showing the strategies for C. scindens colonization in C57BL/ 6J mice (SPF) under basal conditions. 10-week-old male mice were gavaged daily for 15 days (A) or daily for 5 days after pre-treatment with vancomycin (500 mg/L in drinking water) for 7 days (B) with live C. scindens bacteria at 3 different doses (10⁸, 10⁹ or 10¹⁰ CFU) or Vehicle (PBS) (n=4 mice/condition). (C) Quantification of the relative abundance of C. scindens (Cs) by qPCR in feces of mice described in A and B 15 days after the start of the experiment. (D) Shannon diversity index (SDI) showing the fecal bacterial community diversity of SPF under basal conditions. SPF mice pre-treated with vancomycin (500 mg/L in drinking water) for 7 days were gavaged daily for 5 days with live C. scindens (10⁸ CFU-SPF+C. scindens) (n=10 mice) or PBS (control-SPF) (n=11 mice). I Microbiota dysbiosis index of mice described in D. (F) Non-metric multidimensional scaling (NMDS) of amplicon sequence variant (ASV) counts based on the Bray-Curtis dissimilarity of fecal bacterial community diversity of mice described in D. (G) Relative abundance of bacterial Phyla representing more than 10% of total abundance in feces of mice described in D. (H) Pie charts showing composition and amount of BAs in the feces of mice described in D. BA data are normalized to the total amount of BAS. Numbers in the lower left corner of each pie chart correspond to the total amount of BAS (nmol/g of feces). BA species are classified and colored to highlight primary unconjugated (Uncon- . -green), primary conjugated (Con-. -blue), 7α-dehydroxylated unconjugated (7α-DH--d - red), and other secondary unconjugated (purple) BAS. (I) Dot plots showing the BA abundance and the z-score in feces of mice described in D. The dot color represents the z-score for each BA species, whereas the dot size represents the abundance of each BA species over the total BA amount per mouse. 7α-DH-ed BAs are highlighted in red (n=10 mice/condition). (J) Violin plots showing the proportion of primary and secondary BAs (percentage of total BA amount), and the amount of CA, CDCA, DCA and LCA (nmol/g of feces). Graphs represent meantSEM (C, D, E). P values were calculated using one-way analysis of variance (ANOVA) followed by Bonferroni post hoc correction (C, D, E) or 2-tailed Student's t test (J).

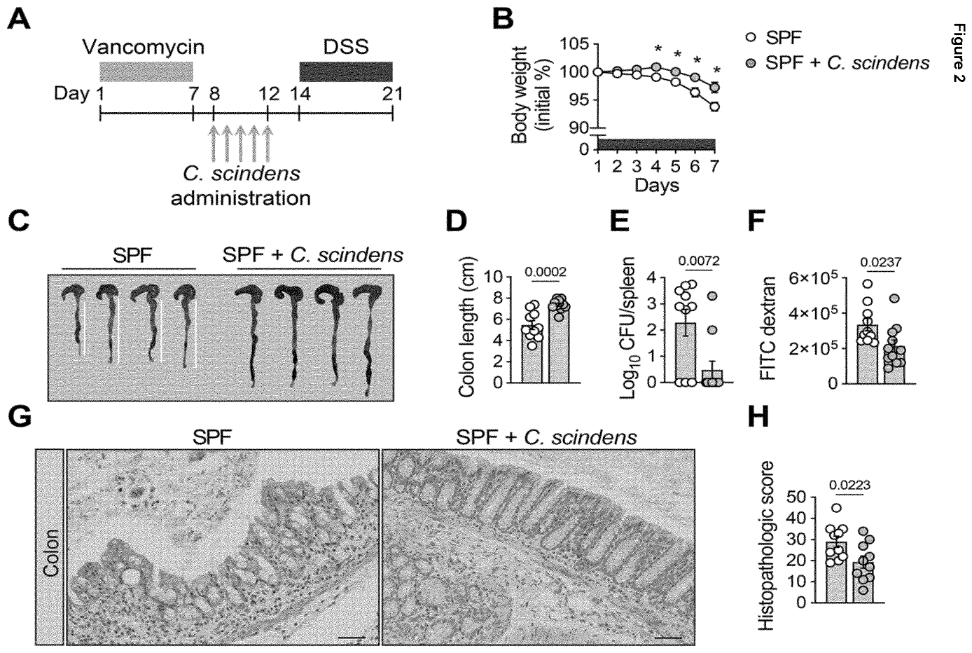

FIG. 2. Administration of C. scindens alleviates DSS-induced colitis and improves intestinal regeneration in conventional SPF mice with microbiota dysbiosis. (A) Workflow of the DSS-induced acute colitis experiment. 10-week-old male C57BL/ 6J (SPF) mice pre-treated with vancomycin (500 mg/L in drinking water) for 7 days were gavaged daily for 5 days with live C. scindens (108 CFU SPF+C. scindens) (n=10 mice) or PBS (control-SPF) (n=11mice). 2 days after colonization, colitis was induced by 7-day treatment with DSS (2.5% in drinking water). (B) Percentage of body weight loss of mice described in A during exposure to DSS. (C and D) Representative images (C) and length (D) of the colon of mice described in A after 7 days of DSS. (E and F) Bacterial CFU in the spleen I and quantification of FITC dextran in plasma (F) of mice described in A at the time of sacrifice (day 7 after the start of DSS). (G) Representative histological images (H&E staining) of the colon of mice described in A after 7 days of DSS administration. (H) Total histopathological score (sum of the following parameters: severity of inflammation, ulceration and crypt damage) calculated on the histological images of the colon of mice described in A after 7 days of DSS. (I) Workflow of the DSS-induced acute colitis experiment. 10-week-old male C57BL/ 6J (SPF) mice pre-treated with vancomycin (500 mg/L in drinking water) for 7 days were gavaged daily for 5 days with live C. scindens (10⁸ CFU-SPF+C. scindens) or PBS (control-SPF) (n=10 mice/condition). 2 days after the colonization, colitis was induced by a 7-day treatment with DSS (2.5% in drinking water) followed by 3 days of drinking water (recovery period). (J) Percentage of body weight loss of mice described in I during DSS exposure and recovery period. (K and L) Representative images (K) and quantification of Edu⁺ or Ki67⁺ cells per crypt (L) in the colon of mice described in I at the time of sacrifice (day 3 after DSS withdrawal). Scale bars=100 μm (G) and 50 μm (K). Graphs represent mean±SEM. P values were calculated using 2-way analysis of variance (ANOVA) followed by Bonferroni post hoc correction (B, J) or 2-tailed Student's t-test (D-F, H, L).

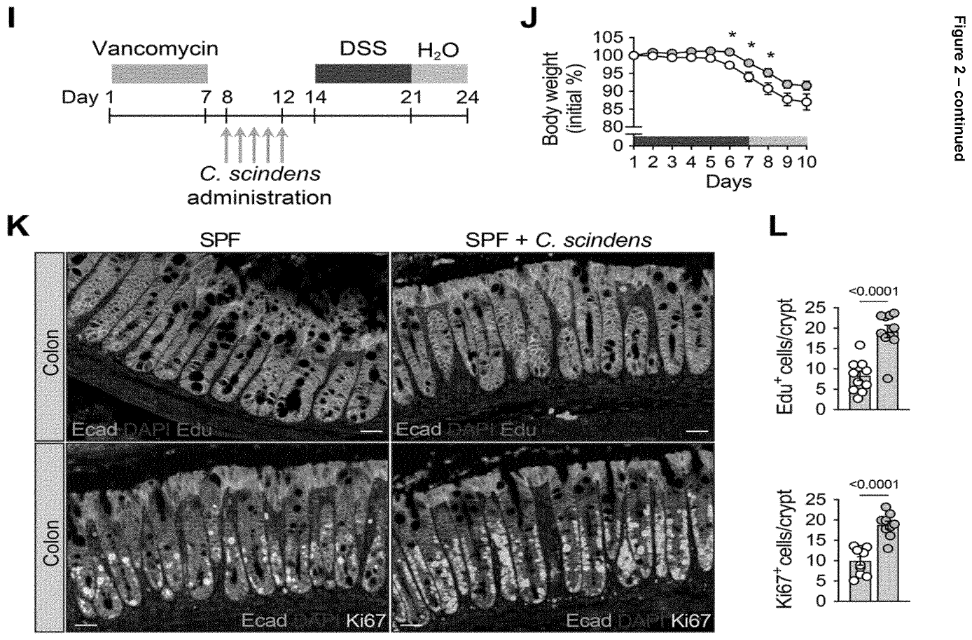

FIG. 3. C. scindens administration ameliorates body weight phenotype in high-fat diet-fed SPF mice. (A) Timeline of the phenotyping protocol. (B) Colonization efficiency (relative abundance) in chow diet (CD)-fed and high-fat diet (HFD)-fed C57BL/6J (SPF) mouse models colonized or not with C. scindens. n=12 mice for SPF CD and SPF CD+C. scindens, n=10 mice for SPF HFD, n=9 mice for SPF HFD+C. scindens.(C) Body weight increase of mice described in B. (D) Fat and lean masses (expressed as % of body weight) quantified by EchoMRI of mice described in I(E) Glycemia levels before and after an oral administration of test meal of mice described in B. (F) Glycemia levels before and after an oral administration of glucose (OGTT) of mice described in B. (G) Energy expenditure over 24 h quantified according to the day/night cycle of mice described in B. (H) Respiratory exchange ratio (RER) over 24 h quantified according to the day/night cycle of mice described in B. Graphs represent mean±SEM (B-H). P values were calculated using one-way analysis of variance (ANOVA) (B, D, G, H) or 2-way analysis of variance (ANOVA) (C, E, F) followed by Tukey post hoc correction.

The examples illustrate the claimed invention.

### EXAMPLE 1

### Human-Derived 7α-Dehydroxylating Bacteria in Combination With Antibiotic Treatment To Promote Intestinal Health

### C. scindens Efficiently Colonizes SPF Mice After Vancomycin-Induced Dysbiosis

We postulated that the addition of 7α-DH-ing bacteria in a dysbiotic microbial community is sufficient to protect mice from chemically-induced colitis. To evaluate this hypothesis, we used a C57BL/6J specific pathogen-free (SPF) mouse model. 10-week-old male mice were gavaged daily with live C. scindens bacteria at 3 different concentrations (10⁸, 10⁹, or 10¹⁰ colony-forming units (CFU)) or with a vehicle (PBS) for 15 days (FIG. 1A) or pre-treated with vancomycin (500 mg/L in drinking water) for 7 days and gavaged daily for 5 days with live C. scindens bacteria at the above doses or vehicle (FIG. 1B). Pre-treatment with vancomycin was chosen to induce microbiota dysbiosis owing to its antimicrobial spectrum against gram-positive bacteria⁷⁰. We conjectured that this treatment might be necessary to clear mucosal microbial niches and allow facilitated engraftment of C. scindens. Quantification of fecal C. scindens abundance in the different experimental conditions revealed that administration of 10⁸ CFU after vancomycin pre-treatment was the optimal condition for intestinal colonization by C. scindens (FIG. 1C). 16S rRNA gene amplicon sequencing was performed to further investigate the effect of vancomycin-induced dysbiosis and C. scindens administration. As expected, vancomycin treatment reduced fecal bacterial community diversity and induced microbiota dysbiosis, as indicated by the Shannon diversity index (SDI) (FIG. 1D) and the dysbiosis index, respectively (FIG. 1E). Notably, these alterations were not compensated by colonization with C. scindens (FIGS. 1D and 1E). In addition, based on the non-metric multidimensional scaling analysis, we observed a separation between mice treated with vehicle or colonized with C. scindens following vancomycin administration (FIG. 1F). Moreover, the bacterial phylum composition was significantly modulated by vancomycin administration, with the emergence of Lachnospiraceae, Akkermansiaceae, Rumminococcaceae and Anaeroplasmataceae phyla and obliteration of the Muribaculaceae phylum (FIG. 1G). Importantly, C. scindens colonization increased fecal 7α-DH-ed BA abundance in dysbiotic, vancomycin-treated SPF mice, while primary BA abundance decreased compared with control SPF mice (FIGS. 1H-J). Overall, these data confirm that C. scindens efficiently colonizes vancomycin-treated SPF mice and is sufficient to augment the 7α-DH-ed BA levels.

### C. scindens Colonization Protects SPF mice with dysbiotic microbiota from DSS-induced acute Colitis

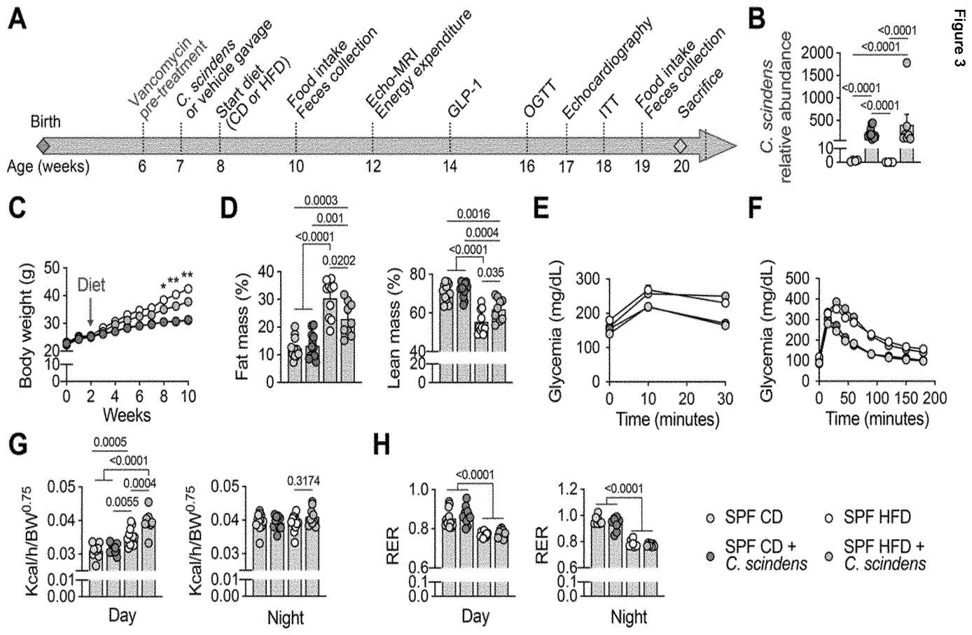

We then subjected this mouse model of dysbiosis to dextran sulfate sodium (DSS)-induced acute colitis (FIG. 2A). DSS is a chemical colitogen with anticoagulant properties widely used to induce epithelial damage in the large intestine⁷¹. We observed that C. scindens colonization protects the SPF mice from DSS-induced acute colitis, as evidenced by reduced body weight loss (FIG. 2B) and increased colon length (FIGS. 2C and 2D). In this context, C. scindens colonization also improved intestinal barrier function, as demonstrated by decreased intestinal bacterial outgrowth in the spleen (FIG. 2E) and decreased diffusion of orally administered FITC dextran into plasma (FIG. 2F), as well as intestinal damage and histopathologic score (FIGS. 2G and 2H). Furthermore, when mice were allowed 3 days of recovery following DSS treatment (FIG. 21), body weight loss remained more pronounced in SPF mice colonized with C. scindens until stabilization of the body weight was reached (FIG. 2J). This protective effect was associated with enhanced regeneration capacity, as indicated by increased proliferation in colonic crypts of SPF mice with dysbiosis colonized with C. scindens, as evidenced by Edu⁺ and Ki67⁺ staining and quantification (FIGS. 2K and 2L).

### EXAMPLE 2

### Use of Human-Derived 7α-Dehydroxylating Bacteria in Combination With Antibiotic Treatment to Promote Metabolic Health

To dissect host-microbiome interactions in the context of BA-TGR5 signaling, our laboratory set out to investigate the metabolic impact of 7α-DH-ing bacteria administration in the context of obesity. We postulated that the addition of 7α-DH-ing bacteria in a high-fat diet (HFD)-induced obesity model would be sufficient to protect mice from metabolic alterations. To test this hypothesis, we developed a new SPF mouse model in which the microbial community was remodeled by antibiotic pre-treatment to free microbial niches of the mucosa, hence allowing optimal C. scindens engraftment.

To evaluate the protective effect of our probiotic treatment in a metabolic context, we colonized 7-week-old male C57BL/ 6J SPF mice with C. scindens or PBS as control (10⁸ CFU daily gavage for 5 days) after an antibiotic pre-treatment with vancomycin for 7 days (500mg/L in drinking water) and fed them chow diet (CD-Safe 150) or HFD (D12492, Research diet) at 8 weeks of age for 12 weeks (FIG. 3A). Comprehensive metabolic phenotyping is still in progress (the mice are 16 weeks old at the time), but below is an overview of the data collected so far.

C. scindens was able to stably colonize SPF mice, and HFD feeding did not affect this ability (FIG. 3B). Interestingly, the mice colonized with C. scindens displayed a lower body weight when fed HFD, but not under CD condition, than the control group (FIG. 3C). These mice furthermore showed lower fat mass and increased lean mass (FIG. 3D), but no difference in the glycemic profile after oral administration of a test meal (FIG. 3E) or a glucose bolus (FIG. 3F). Moreover, HFD-fed SPF mice colonized with C. scindens showed a higher energy expenditure (FIG. 3G), without variation in respiratory exchange ratio (RER) (FIG. 3H), compared to the control group.

These results show the beneficial effect of C. scindens in the context of metabolic disorders and support that C. scindens can be used to stimulate TGR5-dependent metabolic responses in vivo. The above data is based on the age of the mice of currently 16-week-old and the phenotyping will be continued.

### EXAMPLE 3

### Materials and Methods

Gut colonization with C. scindens

Pilot experiment: 10-week-old C57BL/ 6J (Charles River, France) male mice were given daily by oral administration live C. scindens bacteria at 3 different concentrations (10⁸, 10⁹, or 10¹⁰ colony-forming units (CFU)) or vehicle (PBS) for 15 days or pre-treated with vancomycin (500 mg/L in drinking water) for 7 days and gavaged daily for 5 days with live C. scindens bacteria at the above doses or vehicle.

DSS and HFD experiments: 6-week-old (HFD experiment-FIGS. 3) and 10-week-old (DSS experiment—FIG. 2) C57BL/ 6J (Charles River, France) male mice were pre-treated with vancomycin (500 mg/L in drinking water) for 7 days and gavaged daily for 5 days with live C. scindens bacteria (10⁸ CFU) or vehicle.

16S rRNA sequencing: DNA was extracted from fresh mouse feces using a stool DNA kit (Qiagen). The 16S V 3 -V4 region was amplified using 16S rRNA fusion primers. All PCR products were purified by Agencourt AMPure XP beads, dissolved in elution buffer and labeled to finish library construction. Library size and concentration were detected by Agilent 2100 Bioanalyzer. Qualified libraries were sequenced on HiSeq platform (BGI, China) according to their insert size.

Experimental colitis and regeneration assay: colitis was induced in 10-week-old C57BL/ 6J male mice colonized or not with C. scindens after vancomycin pre-treatment by administration of 2.5% dextran sulfate sodium salt (DSS, Sigma 42867) in drinking water. Daily changes in body weight were assessed. Mice were euthanized 7 days after DSS administration. To monitor epithelium regeneration, after 7 days from DSS initiation, DSS was replaced with water for 3 days.

Proliferation assay: cell proliferation was assessed by Edu assay (Click-IT Edu Alexa Fluor 647, ThermoFisher, Waltham, MA) following manufacturer's instructions. Edu was resuspended in phosphate-buffered saline (PBS) and injected intraperitoneally (50 μg per g of mouse weight) 2 hours before euthanasia.

Histology: tissues were fixed with Formal-fixx overnight at 4 °C and embedded in paraffin. Four AM sections were prepared by microtome sectioning (Microm HM325, ThermoFisher, Waltham, MA). Hematoxylin and eosin (Hematoxylin QS; Vector, Eosin Y Solution Aqueous; Sigma) staining was performed according to the manufacturer's protocols. Images were acquired using Olympus (Tokyo, Japan) slide scanner VS120-L100.

Histopathologic scoring: a European board-certified veterinary pathologist performed the histopathologic evaluation in a blinded fashion. The following parameters were identified: (1) severity of inflammation; (2) ulceration; (3) crypt damage. Histologic criteria for each parameter have been adopted and adapted. The sum of the 3 parameter values was used to generate a “total histopathological score”.

Immunohistochemistry and immunofluorescence: antigen retrieval was performed by incubating the colon sections in 10 mM citrate buffer (pH 6.0) for 20 min at 95 °C. After cooling to room temperature, the sections were washed and blocked with blocking buffer (1% BSA (Sigma, A7906) and 0.5% Triton X-100 (Sigma, X100) in PBS) for 1 h at room temperature. The primary antibodies anti-Ki67 (ThermoFisher) and anti-E-cadherin (ThermoFisher) were diluted in a 1:100 dilution of the blocking buffer and incubated overnight at 4 °C. Sections were counterstained with Harris and mounted. For immunofluorescence, sections were washed and incubated for 1 h with Alexa Fluor conjugated secondary antibodies (1:1000 in blocking solution; Invitrogen). Following extensive washing, sections were counterstained with DAPI (ThermoFisher, 62248) and mounted in ProLong Gold Antifade Mountant (Thermo Fischer, P36930). Stained sections were imaged by a virtual slide microscope (VS120, Olympus) and a confocal microscope (SP8, Leica). The image analysis was performed using QuPath software⁷².

BA quantification: the BA measurement was done by stable isotope dilution mass spectrometry assay. Standards for calibration: Stock solutions of BAs and D5-labeled BAs (at 1 mg/mL) in methanol (MeOH) were used for the preparation of standard mixtures with a final concentration from 2.5 to 10 μg/mL. Calibrators were prepared by serial dilutions with stripped serum (C0-C7). Internal standard (IS) mixture contained 13 deuterium-labeled BAS.

Sample preparation: Lyophilized feces were ground with liquid nitrogen using mortar and pestle. Powders were pre-weighed to ~100 mg each in the lysis tubes. Samples were homogenized in tissue homogenizer (Precellys, Bertin Instruments, Montigny-le-bretonneux) by the addition of MeOH: H2O (2:1) with 0.1% formic acid (1500 μL) and ceramic beads for 2×20 seconds at 10,000 rpm. Homogenized extracts were centrifuged for 15 minutes at 14,000 g at 4 °C, and the supernatants were further treated by solid phase extraction (SPE) for phospholipid removal. Before loading onto SPE plate, the s16ogethertogether with the calibrators and QC samples, were spiked with IS mixture and diluted with H2O containing 0.2% formic acid. All plates were sealed, homogenized by vortexing, and centrifuged for 5 minutes at 2700 g at 4 °C. The mixtures were loaded onto an Oasis HLB μElution (Waters, Milford, MA) plate. Once loaded, the plate was washed with 5% MeOH in H2O solution (200 μL) and the analytes were eluted with 100% MeOH (100 μL) after 1 minute of incubation. The eluates were collected and dried under nitrogen flow at 25 °C. The dried extracts were reconstituted in 100 μL of 30% acetonitrile in H2O solution before liquid chromatography high-resolution mass spectrometry (LC-HRMS) analyses.

LC-HRMS analysis: BA quantification was performed on a Vanquish Horizon (ThermoFisher Scientific) ultra-high performance liquid chromatography (UHPLC) system coupled to Q Exactive Focus interfaced with a HESI source operating in negative ionization mode. Chromatographic separation was carried out using an Acquity UPLC HSS T3 1.8 μm 2.1×100 mm column (Waters, Milford, MA). Mass spectrometry parameters were set to full scan in mass range m/z 370-522, mass resolving power=70,000 full with at half maximum and automatic gain control target=5e5.

Data processing: Data were processed using TraceFinder 4.1. software (ThermoFisher Scientific). The linearity was evaluated for each BA. Limit of detection was determined with signal-to-noise (S/N) ratio >3; lower limit of quantification was determined with S/N>5 and <20% coefficient of variation. In addition to accurate m/z ratio, the retention time (in minutes) was used to distinguish between different isobaric species. Absolute concentrations were calculated using calibration curves and considering the response factor of corresponding IS. Concentrations were reported to sample dry weight (feces).

Chow diet and high-fat diet studies: 8-week-old SPF C57BL/ 6J male mice colonized or not with C. scindens were fed a control di-t (CD-SAFE 150, Safe Diets) or a high-fat die—(HFD-D 12492, Research diet) for 12 weeks (ongoing experiment).

C. scindens colonization efficiency: DNA was extracted from fresh fecal pellets and the colonization was confirmed by real-time PCR using primers specific for C. scindens. Data were expressed as relative abundance over 16S.

Body composition analysis (Echo-MRI): body composition analysis was performed by placing each mouse in a magnetic resonance imaging machine (Echo-MRI, the 3-in-1; Echo Medical Systems), where lean and fat masses were recorded. Lean and fat masses were normalized to body weight.

Indirect calorimetry: the indirect calorimetry experiment was performed using the Promethion System (Sable Systems International). Each mouse was individually placed in a cage with normal bedding and free access to food and water during the test, which lasted approximately 48 hours (24 hours of acclimation and 24 hours of measurement). The system was set on a 12/12-hour light/dark cycle. Data were normalized to metabolic body mass (body weight, BWV0.75).

GLP-1 assay: mice were fasted over-day and Ensure Plus (10 ml/Kg) was administered orally at the beginning of the dark phase (7 pm). Blood glucose was determined by ACCU-CHECK glucose meters (Roche) at the indicated time points and blood was collected from the tail vein in EDTA-coated tubes and kept on ice. Plasma was collected by centrifugation and GLP-1 levels will be measured using Luminex technology (Merck Millipore), following manufacturer's instructions.

Oral glucose tolerance test (OGTT): OGTT was performed by measuring blood glucose following oral administration of glucose (2 g/Kg) after overnight fasting. Blood glucose was determined by CONTOUR XT glucose meters (Ascensia Diabetes Care) at the indicated time points. At T0, T15, T30 and T60, blood was collected from the tail vein in Heparin-coated tubes and kept on ice. Plasma was collected by centrifugation and insulin levels will be measured by specific ELISA kits (Crystal Chem), following manufacturer's instructions.

Statistical analysis: statistically significant differences between the means of 2 groups were assessed by unpaired t test, one-or two-way analysis of variance as specified in the legends. All statistical analyses were performed in the GraphPad Prism 9 software. A P value of 0.05 or less was considered statistically significant.

Ethical approval: all mouse experiments were authorized by the Veterinary Office of the Canton of Vaud, Switzerland, under the license authorizations no. 3263.1.

### REFERENCES

1. Krautkramer, K. A., Fan, J. & Backhed, F. Gut microbial metabolites as multi-kingdom intermediates. Nat. Rev. Microbiol. 19, 77-94 (2021).
2. Perino, A., Demagny, H., Velazquez-Villegas, L. A. & Schoonjans, K. Molecular Physiology of Bile Acid Signaling in Health, Disease and Aging. Physiol. Rev. (2020) doi: 10.1152/physrev.00049.2019.
3. Chávez-Talavera, O., Tailleux, A., Lefebvre, P. & Staels, B. Bile Acid Control of Metabolism and Inflammation in Obesity, Type 2 Diabetes, Dyslipidemia, and Nonalcoholic Fatty Liver Disease. Gastroenterology 152, 1679-1694.e3 (2017).
4. Wahlström, A., Sayin, S. I., Marschall, H.-U. & Bäckhed, F. Intestinal Crosstalk between Bile Acids and Microbiota and Its Impact on Host Metabolism. Cell Metab. 24, 41-50 (2016).
5. Collins, S. L., Stine, J. G., Bisanz, J. E., Okafor, C. D. & Patterson, A. D. Bile acids and the gut microbiota: metabolic interactions and impacts on disease. Nat. Rev. Microbiol. (2022) doi: 10.1038/s41579-022-00805-x.
6. Guzior, D. V. & Quinn, R. A. Review: microbial transformations of human bile acids. Microbiome 9, 140(2021 ).
7. Marion, S. et al. In vitro and in vivo characterization of Clostridium scindens bile acid transformations. Gut Microbes 10, 481-503 (2019).
8. Ridlon, J. M. et al. The ‘in vivo lifestyle’ of bile acid 7α-dehydroxylating bacteria: comparative genomics, metatranscriptomic, and bile acid metabolomics analysis of a defined microbial community in gnotobiotic mice. Gut Microbes 11, 381-404 (2020).
9. Funabashi, M. et al. A metabolic pathway for bile acid dehydroxylation by the gut microbiome. Nature 582, 566-570 (2020).
10. Ridlon, J. M., Kang, D.-J. & Hylemon, P. B. Bile salt biotransformations by human intestinal bacteria. J. Lipid Res. 47, 241-259 (2006).
11. Kuipers, F., Bloks, V. W. & Groen, A. K. Beyond intestinal soap—bile acids in metabolic control. Nat. Rev. Endocrinol. 10, 488-498 (2014).
12. Kawamata, Y. et al. A G protein-coupled receptor responsive to bile acids. J. Biol. Chem. 278, 9435-9440 (2003).
13. Pellicciari, R. et al. Discovery of 6alpha-ethyl-23(S)-methylcholic acid (S-EMCA, INT- 777) as a potent and selective agonist for the TGR5 receptor, a novel target for diabesity. J. Med. Chem. 52, 7958-7961 (2009).
14. Thomas, C. et al. TGR5-mediated bile acid sensing controls glucose homeostasis. Cell Metab. 10, 167-177 (2009).
15. Watanabe, M. et al. Bile acids induce energy expenditure by promoting intracellular thyroid hormone activation. Nature 439, 484-489 (2006).
16. Chen, X. et al. Chenodeoxycholic acid attenuates high-fat diet-induced obesity and hyperglycemia via the G protein-coupled bile acid receptor 1 and proliferator-activated receptor γ pathway. Exp. Ther. Med. 14, 5305-5312 (2017).
17. Carino, A. et al. Agonism for the bile acid receptor GPBAR 1 reverses liver and vascular damage in a mouse model of steatohepatitis. FASEB J. Off. Publ. Fed. Am. Soc. Exp. Biol. 33, 2809-2822 (2019).
18. Briere, D. A. et al. Novel Small Molecule Agonist of TGR5 Possesses Anti-Diabetic Effects but Causes Gallbladder Filling in Mice. PloS One 10, e0136873 (2015).
19. Zietak, M. & Kozak, L. P. Bile acids induce uncoupling protein 1-dependent thermogenesis and stimulate energy expenditure at thermoneutrality in mice. Am. J. Physiol. Endocrinol. Metab. 310, E346-354 (2016).
20. Zheng, X. et al. Hyocholic acid species improve glucose homeostasis through a distinct TGR5 and FXR signaling mechanism. Cell Metab. 33, 791-803.e7 (2021).
21. Holter, M. M. et al. Compound 18 Improves Glucose Tolerance in a Hepatocyte TGR5-dependent Manner in Mice. Nutrients 12, 2124 (2020).
22. Castellanos-Jankiewicz, A. et al. Hypothalamic bile acid-TGR5 signaling protects from obesity. Cell Metab. 33, 1483-1492.e10 (2021).
23. Broeders, E. P. M. et al. The Bile Acid Chenodeoxycholic Acid Increases Human Brown Adipose Tissue Activity. Cell Metab. 22, 418-426 (2015).
24. Velazquez-Villegas, L. A. et al. TGR5 signalling promotes mitochondrial fission and beige remodelling of white adipose tissue. Nat. Commun. 9, 245(2018 ).
25. Carino, A. et al. Gpbar 1 agonism promotes a Pgc-1α-dependent browning of white adipose tissue and energy expenditure and reverses diet-induced steatohepatitis in mice. Sci. Rep. 7, 13689 (2017).
26. Perino, A. et al. TGR5 reduces macrophage migration through mTOR-induced C/EBPβ differential translation. J. Clin. Invest. 124, 5424-5436 (2014).
27. Eggink, H. M. et al. Chronic infusion of taurolithocholate into the brain increases fat oxidation in mice. J. Endocrinol. 236, 85-97 (2018).
28. Sasaki, T. et al. The exercise-inducible bile acid receptor Tgr5 improves skeletal muscle function in mice. J. Biol. Chem. 293, 10322-10332 (2018).
29. Sasaki, T. et al. Muscle-specific TGR5 overexpression improves glucose clearance in glucose-intolerant mice. J. Biol. Chem. 296, 100131(2021 ).
30. Brighton, C. A. et al. Bile Acids Trigger GLP-1 Release Predominantly by Accessing Basolaterally Located G Protein-Coupled Bile Acid Receptors. Endocrinology 156, 3961-3970 (2015).
31. Kuhre, R. E. et al. Bile acids are important direct and indirect regulators of the secretion of appetite-and metabolism-regulating hormones from the gut and pancreas. Mol. Metab. 11, 84-95 (2018).
32. Calderon, G. et al. Ileo-colonic delivery of conjugated bile acids improves glucose homeostasis via colonic GLP-1-producing enteroendocrine cells in human obesity and diabetes. EBioMedicine 55, 102759 (2020).
33. Harach, T. et al. TGR5 potentiates GLP-1 secretion in response to anionic exchange resins. Sci. Rep. 2, 430(2012 ).
34. Sorrentino, G. et al. Bile Acids Signal via TGR5 to Activate Intestinal Stem Cells and Epithelial Regeneration. Gastroenterology 159, 956-968.e8 (2020).

35. Lund, M. L. et al. L-Cell Differentiation Is Induced by Bile Acids Through GPBAR1 and Paracrine GLP-1 and Serotonin Signaling. Diabetes 69, 614-623 (2020).
36. Gribble, F. M. & Reimann, F. Metabolic Messengers: glucagon-like peptide 1. Nat. Metab. 3, 142-148 (2021).
37. Hammoud, R. & Drucker, D. J. Beyond the pancreas: contrasting cardiometabolic actions of GIP and GLP1. Nat. Rev. Endocrinol. (2022) doi: 10.1038/s41574-022-00783-3.
38. Perino, A. et al. Central anorexigenic actions of bile acids are mediated by TGR5. Nat. Metab. 3, 595-603 (2021).
39. Perino, A. & Schoonjans, K. Metabolic Messengers: bile acids. Nat. Metab. 4, 416-423 (2022).
40. Sun, H. et al. IDF Diabetes Atlas: Global, regional and country-level diabetes prevalence estimates for 2021 and projections for 2045. Diabetes Res. Clin. Pract. 183, 109119(2022 ).
41. Schnurr, T. M. et al. Obesity, unfavourable lifestyle and genetic risk of type 2 diabetes: a case-cohort study. Diabetologia 63, 1324-1332 (2020).
42. Lingvay, I., Sumithran, P., Cohen, R. V. & le Roux, C. W. Obesity management as a primary treatment goal for type 2 diabetes: time to reframe the conversation. Lancet Lond. Engl. 399, 394-405 (2022).
43. Sinclair, P., Brennan, D. J. & le Roux, C. W. Gut adaptation after metabolic surgery and its influences on the brain, liver and cancer. Nat. Rev. Gastroenterol. Hepatol. 15, 606-624 (2018).
44. Seeley, R. J., Chambers, A. P. & Sandoval, D. A. The role of gut adaptation in the potent effects of multiple bariatric surgeries on obesity and diabetes. Cell Metab. 21, 369-378 (2015).
45. Gribble, F. M. & Reimann, F. Function and mechanisms of enteroendocrine cells and gut hormones in metabolism. Nat. Rev. Endocrinol. 15, 226-237 (2019).
46. Sonnenburg, J. L. & Bäckhed, F. Diet-microbiota interactions as moderators of human metabolism. Nature 535, 56-64 (2016).
47. Gentile, C. L. & Weir, T. L. The gut microbiota at the intersection of diet and human health. Science 362, 776-780 (2018).
48. Aron-Wisnewsky, J., Warmbrunn, M. V., Nieuwdorp, M. & Clément, K. Metabolism and Metabolic Disorders and the Microbiome: The Intestinal Microbiota Associated With Obesity, Lipid Metabolism, and Metabolic Health-Pathophysiology and Therapeutic Strategies. Gastroenterology 160, 573-599 (2021).
49. Qin, J. et al. A metagenome-wide association study of gut microbiota in type 2 diabetes. Nature 490, 55-60 (2012).
50. Ng, S. C. et al. Microbiota engraftment after faecal microbiota transplantation in obese subjects with type 2 diabetes: a 24-week, double-blind, randomised controlled trial. Gut 71, 716-723 (2022).
51. Guan, Q. A Comprehensive Review and Update on the Pathogenesis of Inflammatory Bowel Disease. J. Immunol. Res. 2019, 1-16 (2019).
52. Jiang, P., Wu, S., Luo, Q., Zhao, X.-M. & Chen, W.-H. Metagenomic Analysis of Common Intestinal Diseases Reveals Relationships among Microbial Signatures and Powers Multidisease Diagnostic Models. mSystems 6, e00112-21 (2021).
53. Lloyd-Price, J. et al. Multi-omics of the gut microbial ecosystem in inflammatory bowel diseases. Nature 569, 655-662 (2019).
54. Duboc, H. et al. Connecting dysbiosis, bile-acid dysmetabolism and gut inflammation in inflammatory bowel diseases. Gut 62, 531-539 (2013).
55. Graham, D. B. & Xavier, R. J. Pathway paradigms revealed from the genetics of inflammatory bowel disease. Nature 578, 527-539 (2020).
56. Baumgart, D. C. & Carding, S. R. Inflammatory bowel disease: cause and immunobiology. The Lancet 369, 1627-1640 (2007).
57. Williams, J. M. et al. Epithelial cell shedding and barrier function: a matter of life and death at the small intestinal villus tip. Vet. Pathol. 52, 445-455 (2015).
58. Peterson, L. W. & Artis, D. Intestinal epithelial cells: regulators of barrier function and immune homeostasis. Nat. Rev. Immunol. 14, 141-153 (2014).
59. Rogler, G. Resolution of inflammation in inflammatory bowel disease. Lancet Gastroenterol. Hepatol. 2, 521-530 (2017).
60. Saeid Seyedian, S. et al. A review of the diagnosis, prevention, and treatment methods of inflammatory bowel disease. J. Med. Life 12, 113-122 (2019).
61. Villablanca, E. J., Selin, K. & Hedin, C. R. H. Mechanisms of mucosal healing: treating inflammatory bowel disease without immunosuppression? Nat. Rev. Gastroenterol. Hepatol. (2022) doi: 10.1038/s41575-022-00604-y.
62. Song, Z. et al. Taxonomic profiling and populational patterns of bacterial bile salt hydrolase (BSH) genes based on worldwide human gut microbiome. Microbiome 7, 9 (2019).
63. Sokol, H. et al. Faecalibacterium prausnitzii is an anti-inflammatory commensal bacterium identified by gut microbiota analysis of Crohn disease patients. Proc. Natl. Acad. Sci. 105, 16731-16736 (2008).
64. Quévrain, E. et al. Identification of an anti-inflammatory protein from Faecalibacterium prausnitzii, a commensal bacterium deficient in Crohn's disease. Gut 65, 415-425 (2016).
65. Cao, Y., Shen, J. & Ran, Z. H. Association between Faecalibacterium prausnitzii Reduction and Inflammatory Bowel Disease: A Meta-Analysis and Systematic Review of the Literature. Gastroenterol. Res. Pract. 2014, 872725(2014 ).
66. Touch, S. et al. Human CD4+CD8α+ Tregs induced by Faecalibacterium prausnitzii protect against intestinal inflammation. JCI Insight 7, e154722 (2022).
67. Lopez-Siles, M. et al. Changes in the Abundance of Faecalibacterium prausnitzii Phylogroups I and Il in the Intestinal Mucosa of Inflammatory Bowel Disease and Patients with Colorectal Cancer. Inflamm. Bowel Dis. 22, 28-41 (2016).
68. Duncan, S. H., Hold, G. L., Harmsen, H. J. M., Stewart, C. S. & Flint, H. J. Growth requirements and fermentation products of Fusobacterium prausnitzii, and a proposal to reclassify it as Faecalibacterium prausnitzii gen. nov., comb. nov. Int. J. Syst. Evol. Microbiol. 52, 2141-2146 (2002).
69. Heinken, A. et al. Systematic assessment of secondary bile acid metabolism in gut microbes reveals distinct metabolic capabilities in inflammatory bowel disease. Microbiome 7, 75 (2019).
70. Watanakunakorn, C. Mode of action and in-vitro activity of vancomycin. J. Antimicrob. Chemother. 14 Suppl D, 7-18 (1984).
71. Chassaing, B., Aitken, J. D., Malleshappa, M. & Vijay-Kumar, M. Dextran sulfate sodium (DSS)-induced colitis in mice. Curr. Protoc. Immunol. 104, 15.25.1-15.25.14 (2014).
72. Bankhead, P. et al. QuPath: Open source software for digital pathology image analysis. Sci. Rep. 7, 16878(2017 ).

1. One or more antibiotic(s) that disrupt(s) the microbial balance of the gut and a microorganism having bile acid 7α-dehydroxylation activity for use in the treatment of a non-infectious disease in a subject.

2. The one or more antibiotic(s) and the microorganism for use of claim 1, wherein the one or more antibiotic(s) is/are to be administered in a pre-treatment to disrupt the microbial balance of the gut before the microorganism having bile acid 7α-dehydroxylation activity is to be administered to the subject.

3. The one or more antibiotic(s) and the microorganism for use of claim 2, wherein the one or more antibiotic(s) is/are to be administered to disrupt the microbial balance of the gut for at least 1 day, preferably at least 3 days and most preferably at least 7 days before the microorganism having bile acid 7α-dehydroxylation activity is to be administered, preferably orally.

4. The one or more antibiotic(s) and the microorganism for use of any one of claims 1 to 3, wherein the one or more antibiotic(s) is/are selected from antibiotics against gram-positive bacteria, preferably from antibiotics being effective against Clostridioides difficile, and most preferably from vancomycin, fidaxomicin, and metronidazole.

5. The one or more antibiotic(s) and the microorganism for use of any one of claims 1 to 4, wherein the one or more antibiotic(s) is/are to be administered
(a) in an amount of 6 to 10 mg/kg, preferably 7 to 8.5 mg/kg of body weight of the subject to be treated, or
(b) in an amount of 100 to 150 mg, most preferably about 125 mg 4 times daily, preferably orally.

6. The one or more antibiotic(s) and the microorganism for use of any one of the preceding claims, wherein the microorganism is a bacterium, preferably a bacterium of the class Clostridia and most preferably Clostridium scindens, Clostridium hylemonae, Peptacetobacter (Clostridium) hiranonis, or Proteocatella sphenisci.

7. The one or more antibiotic(s) and the microorganism for use of claim 6, wherein bacterium is in a proliferative state, an attenuated state or is a bacterial spore.

8. The one or more antibiotic(s) and the microorganism for use of any one of the preceding claims, wherein the non-infectious disease is an inflammatory disease or a metabolic disease.

9. The one or more antibiotic(s) and the microorganism for use of claim 8, wherein the inflammatory disease is inflammatory bowel disease, and is preferably Crohn's disease and/or ulcerative colitis.

10. The one or more antibiotic(s) and the microorganism for use of claim 8, wherein the metabolic disease is obesity, metabolic dysfunction-associated fatty liver disease (MAFLD) or diabetes, wherein the diabetes is preferably type-2 diabetes.

11. The one or more antibiotic(s) and the microorganism for use of any one of the preceding claims, wherein the microorganism having bile acid 7α-dehydroxylation activity is to be administered in an amount of between 10⁵ and 10⁹ colony forming units (CFUs), preferably 10⁷ and 10⁹ CFUs per day and most preferably about 10⁸ CFUs per day, preferably orally or rectally.

12. The one or more antibiotic(s) and the microorganism for use of any one of the preceding claims, wherein the microorganism having bile acid 7α-dehydroxylation activity is to be administered for at least 1 days, preferably at least 2 days, more preferably at least 3 days, even more preferably at least 4 days and most preferably at least 5 days, preferably orally or rectally.

13. The one or more antibiotic(s) and the microorganism for use of any one of the preceding claims, wherein the microorganism having bile acid 7α-dehydroxylation activity is to be administered together with a microorganism having bile salt hydrolase activity, wherein the microorganism having bile salt hydrolase activity is preferably a bacterium, more preferably a bacterium of the genus Faecalibacterium, Bacteroides, Lactococcus or Mediterraneibacter, and most preferably Faecalibacterium prausnitzii, Bacteroides dorei, Lactococcus lactis, Faecalibaculum rodentium or Ruminococcus gnavus.

14. The one or more antibiotic(s) and the microorganism for use of any one of the preceding claims, wherein the subject to be treated is a mammal, preferably human.
